(19) United States
(12) Patent Application Publication
(10) Pub. No.: US 2012/0065373 A1
(43) Pub. Date: Mar. 15, 2012

(54) DENGUE VACCINE, PHARMACEUTICAL COMPOSITION COMPRISING THE SAME, AND NUCLEOTIDE SEQUENCE

(75) Inventor: Yee-Shin LIN, Tainan City (TW)

(73) Assignee: National Cheng Kung University, Tainan City (TW)

(21) Appl. No.: 13/230,273

(22) Filed: Sep. 12, 2011

(30) Foreign Application Priority Data
Sep. 13, 2010 (TW) 099130861

(51) Int. Cl. C07K 14/18; C07H 21/04

(52) U.S. Cl. 530/350; 536/23.72

(57) ABSTRACT

The present invention relates to a dengue vaccine, a pharmaceutical composition including the same, and a nucleotide sequence. The dengue vaccine includes N and C termini-deleted nonstructural protein ΔNC NS1 with a peptide fragment from amino acids 36 to 270, which is derived from dengue virus nonstructural protein 1 (DV NS1) with deletions of N-terminal region from amino acids 1 to 35 and C-terminal region from amino acids 271 to 352. The dengue vaccine of the present invention is depleted of cross-reactivity with endothelial cells and platelets, and can shorten the bleeding time.

### CROSS REFERENCE TO RELATED APPLICATION

This application claims the benefits of the Taiwan Patent Application Serial Number 099130861, filed on Sep. 13, 2010, the subject matter of which is incorporated herein by reference.

### BACKGROUND OF THE INVENTION

1. Field of the Invention

The present invention relates to a dengue vaccine, a pharmaceutical composition comprising the same and a nucleotide sequence, more particularly, to a dengue vaccine with reduced cross-reactivity with endothelial cells and platelets, a pharmaceutical composition comprising the same, and a nucleotide sequence.

2. Description of Related Art

Dengue fever, also called breakbone fever, is an acute infectious disease induced by the propagation of dengue viruses (DV) via Aedes aegypti or Aedes albopictus and its symptoms include high fever (39° C. to 40° C.) or aversion to cold, skin rash, fatigue in limbs, muscle pain, frontal headache, retro-orbital pain, abdominal pain, backache (hence the term breakbone fever), sore throat, and maybe vomiting and fainting. The commonly mentioned dengue virus is classic dengue fever, also called primary dengue fever. In addition, severe and life-threatening dengue fever characterized by hemorrhage or shock may be developed, also called dengue hemorrhagic fever (DHF) or dengue shock syndrome (DSS), or secondary dengue.

It is estimated that there are about 50 million to 100 million cases of dengue infection worldwide each year, with about 250,000 to 500,000 cases of dengue hemorrhagic fever. In other words, two-fifths of the world's population is at risk from dengue infection. Hence, the prevention and treatment of dengue fever is an important issue for the governments of many countries. However, the mechanism underlying dengue disease is not clearly understood. Furthermore, neither effective and safe vaccines nor drugs on specific treatment of diseases caused by dengue viruses are available so far. Since dengue virus is the major pathogen of dengue disease, the early detection or prevention with effective vaccine can efficiently control morbidity and death rates of dengue fever.

Recently, numerous strategies of dengue vaccine design are based on the neutralizing efficacy of antibodies against viral envelope (E) or nonstructure (NS) protein. Although the E protein is responsible for eliciting major neutralizing antibodies during DV infection, it is also associated with the induction of antibody dependent enhancement (ADE). Antibody dependent enhancement (ADE) is a complicating factor in dengue vaccine development in that the pre-existing antibodies raise concerns of causing more severe disease. In contrast, antibodies against the NS1 protein are able to kill DV-infected cells by complement-induced cell lysis and thus are not associated with ADE. A limitation to the vaccine strategy of NS1, however, is that anti-NS1 antibodies may cause cross-reaction with endothelial cells and platelets, and thus may negatively influence coagulation function, resulting in prolonged bleeding time. The side effect of autoimmunity caused by the vaccine still has not been resolved.

Therefore, it is desirable for the technical field to develop a novel vaccine which does not induce antibody dependent enhancement and autoimmunity, is depleted of cross-reactivity with endothelial cells and platelets, and is able to shorten bleeding time.

### SUMMARY OF THE INVENTION

The dengue vaccine according to the present invention includes C and N termini-deleted nonstructural protein ΔNC NS1 with a peptide fragment from amino acids 36 to 270, which is derived from dengue virus nonstructural protein 1 (DV NS1) with deletions of N-terminal region from amino acids 1 to 35 and C-terminal region from amino acids 271 to 352.

Through sequence alignment analysis, the inventors of the present invention found that the N-terminal and C-terminal regions of DV NS1 protein contain epitopes involved in cross-reactivity to target proteins. Therefore, the inventors of the present invention used N terminus (aa 1-35)-deleted and C terminus (aa 271-352)-deleted DV ΔNC NS1 protein as a vaccine to reduce the cross-reactivity with endothelial cells and platelets and thus to shorten bleeding time. In addition, DV ΔNC NS1 protein of the present invention is derived from dengue virus nonstructural protein rather than dengue virus envelope protein, and thus can be used as an inventive and practical vaccine without antibody dependent enhancement and autoimmunity.

In the dengue vaccine of the present invention, the N and C termini-deleted nonstructural protein ΔNC NS1 preferably has the amino acid sequence of SEQ. ID. NO. 1.

In the dengue vaccine of the present invention, the N and C termini-deleted nonstructural protein ΔNC NS1 preferably is derived from dengue virus protein. The dengue virus protein is, for example, SEQ. ID. NO. 3 in the sequence listing.

In the dengue vaccine of the present invention, the dengue vaccine preferably is used to prevent dengue hemorrhagic fever or dengue shock syndrome.

In the dengue vaccine according to the present invention, the amino acid sequence similarity between the N and C termini-deleted nonstructural protein ΔNC NS1 and the SEQ. ID. NO. 1 preferably is 90% or more, and more preferably is 95% or more.

The present invention further provides a dengue vaccine-containing pharmaceutical composition. The dengue vaccine includes N and C termini-deleted nonstructural protein ΔNC NS1 with a peptide fragment from amino acids 36 to 270, which is derived from dengue virus nonstructural protein 1 (DV NS1) with deletions of N-terminal region from amino acids 1 to 35 and C-terminal region from amino acids 271 to 352.

The dengue vaccine-containing pharmaceutical composition according to the present invention includes N terminus (aa 1-35)-deleted and C terminus (aa 271-352)-deleted DV ΔNC NS1 protein, which is depleted of cross-reactivity with endothelial cells and platelets, is able to shorten bleeding time and does not cause antibody dependent enhancement and autoimmunity.

In the dengue vaccine-containing pharmaceutical composition according to the present invention, the N and C termini-deleted nonstructural protein ΔNC NS1 preferably has the amino acid sequence of SEQ. ID. NO. 1.

The dengue vaccine-containing pharmaceutical composition according to the present invention preferably is used to treat or prevent dengue hemorrhagic fever or dengue shock syndrome.

In the dengue vaccine-containing pharmaceutical composition according to the present invention, the amino acid sequence similarity between the N and C termini-deleted nonstructural protein ΔNC NS1 and the SEQ. ID. NO. 1 preferably is 90% or more, and more preferably is 95% or more.

The present invention also provides a nucleotide sequence that encodes N and C termini-deleted nonstructural protein ΔNC NS1. Herein, the N and C termini-deleted nonstructural protein ΔNC NS1 contains DV NS1 from amino acids 36 to 270.

The N and C termini-deleted nonstructural protein ΔNC NS1 encoded from the nucleotide sequence of the present invention is able to act as a dengue vaccine and shorten bleeding time, depleted of cross-reactivity with endothelial cells and platelets, and does not cause antibody dependent enhancement and autoimmunity.

The nucleotide sequence of the present invention preferably is SEQ. ID. NO. 2 in the sequence listing. Preferably, SEQ. ID. NO. 2 is obtained from the nucleotide sequence of dengue virus, which is shown as SEQ. ID. NO. 4 in the sequence listing.

The nucleotide sequence of the present invention preferably is used to produce a dengue vaccine.

### BRIEF DESCRIPTION OF THE DRAWINGS

FIG. 1 shows the results of antibody binding to platelet assay in vitro according to one preferred example of the present invention;

FIG. 2 shows the results of antibody binding to platelet assay on passively immunized mice according to one preferred example of the present invention;

FIG. 3 shows the results of bleeding time tests on actively immunized mice according to one preferred example of the present invention; and

FIG. 4 shows the results of antibody binding to endothelial cell assay in vitro according to one preferred example of the present invention.

### DETAILED DESCRIPTION OF THE PREFERRED EMBODIMENT

### Mice

C3H/HeN mice were obtained from The Jackson Laboratory and maintained on standard laboratory food and water in the Laboratory Animal Center of National Cheng Kung University Medical College. Their 8-wk-old progeny were used for the experiments. Housing, breeding, and experimental use of the animals were performed in strict accordance with the Experimental Animal Committee in National Cheng Kung University.

### [Platelet Preparation]

Human whole blood containing anticoagulant (29.9 mM sodium citrate, 113.8 mM glucose, 72.6 mM NaCl, and 2.9 mM citric acid (pH 6.4)) was centrifuged at 100×g for 20 min at room temperature to obtain platelet-rich plasma (PRP). The platelet-rich plasma was centrifuged at 1000×g for 10 min at room temperature and washed in EDTA/PBS buffer twice. The washed platelets were suspended in Tyrode's solution (137 mM NaCl, 20 mM HEPES, 3.3 mM NaH₂PO₄, 2.4 mM KCl, 1 mg/ml BSA, and 5.6 mM glucose (pH 7.4)) at a concentration of 10⁸ platelets/ml.

[cDNA]

Dengue type 2 (New Guinea C strain) virus grown in BHK21 cells was available from National Institute of Preventive Medicine, Department of Health, and cDNA were obtained. Escherichia coli BL-21 (DE3) pLysS strain bearing the plasmid pRSET-DVNS1 was used to express recombinant proteins of DV NS1 and was available from Shiau's lab (Dr. Ai-Li Shiau) of Department of Microbiology and Immunology of National Cheng Kang University (Taiwan).

[DV ΔNC NS1 from cDNA]

Primers P3758, P3759, P3760 and P3761 were used to amplify DV ΔNC NS1 cDNA from the pPRSET-DVNS1. Herein, PCR was performed as follows.

[symbol]

Primer [symbol]
P3758: 5′-AATTCCCAGAATCCCCTTCAAAACTG-3′;
P3759: 5′-CCCAGAATCCCCTTCAAAACTG-3;
P3760: 5′-TCGAGTCATAAATGCCATGGTCC-3′;
P3761: 5′-GTCATAAATGCCATGGTCCTGCTAT-3′.

PCR primers, pPRSET-DVNS1 as a template and reaction enzymes (including DNA polymerase, T4 polynucleotide kinase and deoxymononucleotide) were placed into two centrifuge tubes, respectively, and uniformly mixed to perform a denaturation step (95° C. for 5 min) and a renaturation step (65° C. for 10 min). About 25% of PCR products were DV ΔNC NS1 cDNAs with cohesive ends containing EcoRI and XhoII restriction sites. The ligation was accomplished by insertion of PCR products into a vector (pET28a).

### [Recombinant Protein and Antibody Preparation]

JEV NS1, DV NS1, and N terminus (aa 1-35)-deleted and C terminus (aa 271-352)-deleted dengue virus nonstructural protein 1 (DV ΔNC NS1) cDNA were cloned into the above-mentioned vector with histidine-tag. Plasmids were introduced into Escherichia coli BL21. The recombinant proteins were induced by 1 μM isopropyl B-D-1-thiogalactopyranoside (Calbiochem) and purified with Ni²⁺ columns. Subsequently, proteins were examined using 10% SDS-PAGE. Proteins from SDS-PAGE were excised and homogenized in adjuvant to immunize mice. Purified protein (25 μg) was emulsified in CFA for the first immunization, and 2 wk later in IFA for 2, 3 or 4 immunizations every week. Mouse sera were collected 3 days after the last immunization, and IgG was purified using protein G columns (Pharmacia Fine Chemicals).

### [Antibody Binding to Platelet Assay]

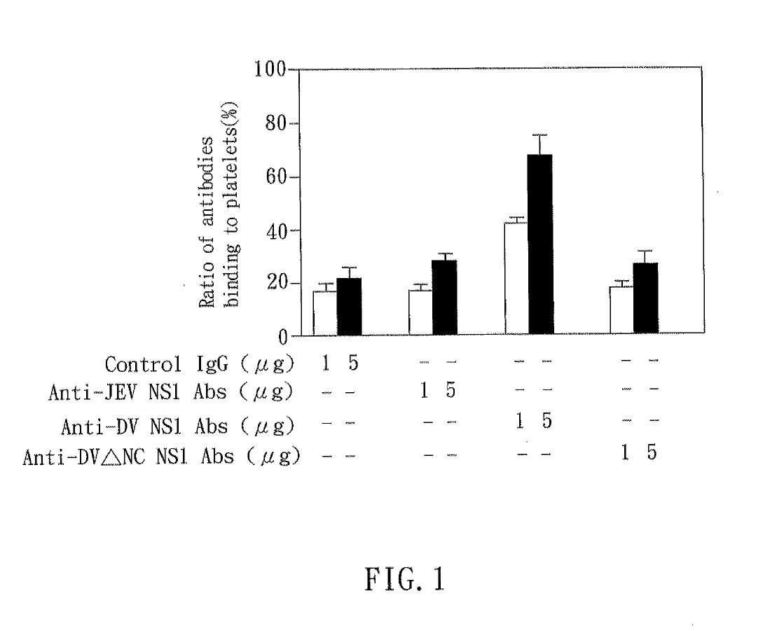

Washed platelets were fixed with 1% formaldehyde in PBS at room temperature for 10 min and then washed with PBS. Various doses of anti-full-length DV NS1, anti-DV ΔNC NS1 or anti-JEV NS1 were incubated with platelets for 30 min. After washing, platelets were incubated with FITC-conjugated anti-mouse IgG (Jackson ImmunoResearch Laboratories) for 30 min. Antibody binding to platelets was analyzed using flow cytometry, as shown in FIG. 1.

As shown in FIG. 1, the binding ability of anti-DV ΔNC NS1 antibody to human platelets was lower than that of anti-full-length DV NS1 antibody, and similar to that of control IgG and anti-JEV NS1.

### [Antibody Binding to Platelet Assay—Animal Model]

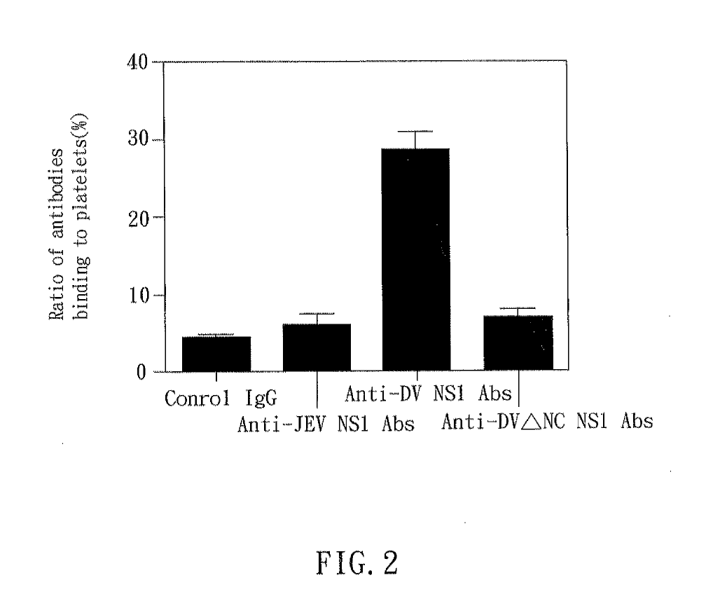

The antibody binding to platelet assay was performed on passively immunized mice with IgG (as a control), anti-full-length DV NS1, anti-DV ΔNC NS1 or anti-JEV NS1 Abs. The results are shown in FIG. 2.

It can be found that the binding ability of anti-DV ΔNC NS1 antibody to platelets was significantly lower than that of anti-full-length DV NS1 antibody, and similar to that of control IgG and anti-JEV NS1.

### [Bleeding Time]

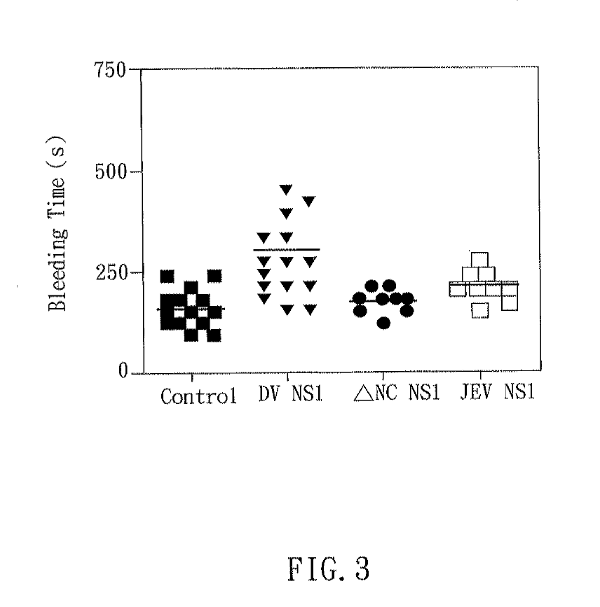

Actively immunized mice with full-length DV NS1, DV ΔNC NS1 and JEV NS1 proteins and normal control mice with no above-mentioned proteins were tested on bleeding time. Bleeding time was performed by a 3-mm tail-tip transection. Blood droplets were collected on filter paper every 30 s for the first 3 min, and every 10 s thereafter. Bleeding time was recorded when the blood spot was smaller than 0.1 mm in diameter. The results are shown in FIG. 3.

As shown in FIG. 3, the bleeding time in N terminus (aa 1-35)-deleted and C terminus (aa 271-352)-deleted dengue virus nonstructural protein 1 (DV ΔNC NS1)-immunized mice was shorter than that in full-length DV NS1-immunized mice, and similar to that in normal control mice and JEV NS1-immunized mice. The results show that the dengue vaccine including N terminus (aa 1-35)-deleted and C terminus (aa 271-352)-deleted dengue virus nonstructural protein 1 (DV ΔNC NS1) according to the present invention indeed has the effect for shortening bleeding time.

### [Antibody Binding to Endothelial Cell Assay]

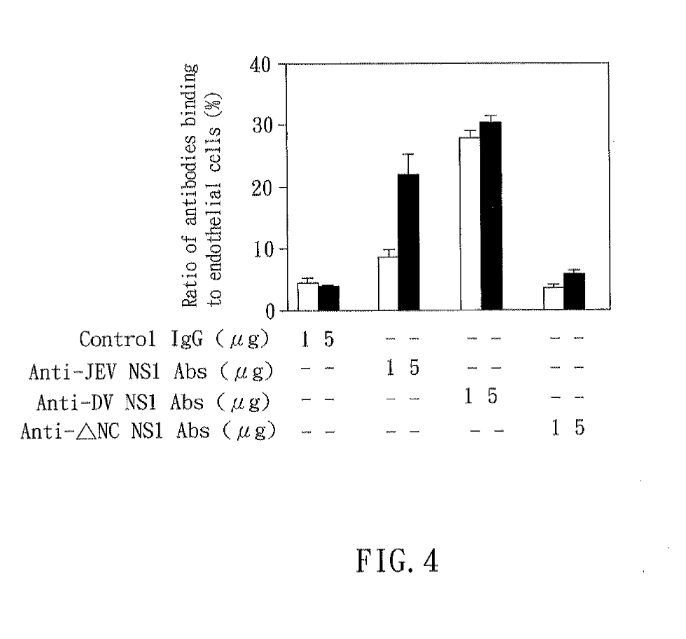

The human microvascular endothelial cell line (HMEC-1) was available from Center for Disease Control and Prevention, Atlanta, Ga. The cells were cultured at 37° C. and washed by PBS, followed by the addition of trypsin-EDTA, and reacted for several minutes. Then, the cells were re-suspended in a fresh serum-containing medium in an appropriate amount. After centrifugation, the cells were fixed with 1% formaldehyde in PBS for 10 min and then washed with PBS. Diluted antibodies in an appropriate dose were incubated with the cells for 1 hr. After washing with PBS for three times, the cells were incubated with FITC-conjugated secondary antibodies for 1 hr. The amount of antibody binding to endothelial cells was analyzed using flow cytometry, as shown in FIG. 4.

The results show that the binding ability of the anti-DV ΔNC NS1 antibody according to the present invention to endothelial cells was significantly lower than that of anti-full-length DV NS1 antibody, and similar to that of IgG antibody (as a control) and anti-JEV NS1 antibody. Therefore, it can be confirmed that the anti-DV ΔNC NS1 antibody according to the present invention does not cause autoimmunity.

Through sequence alignment analysis, the inventors of the present invention found that the N-terminal and C-terminal regions of DV NS1 protein contain epitopes involved in cross-reactivity to target proteins. Therefore, the present invention evaluated and compared the influence of the full-length DV NS1 protein and the N terminus (aa 1-35)-deleted and C terminus (aa 271-352)-deleted DV ΔNC NS1 on cross-reactivity and bleeding time. The experimental results showed that the binding ability of anti-DV ΔNC NS1 antibody to endothelial cells and platelets was lower than that of anti-full-length DV NS1 antibody. Through mouse models (animal models), it was found that the actively immunized mice with the full-length DV NS1 protein showed prolonged bleeding time, while the phenomenon was not found in the immunized mice with the DV ΔNC NS1 protein. The tests on passively immunized mice proved that the anti-DV ΔNC NS1 antibody showed lower binding ability to platelets and thus had better effect for the reduction of bleeding time compared to the anti-full-length DV NS1 antibody.

In conclusion, the vaccine including N and C termini-deleted DV ΔNC NS1 protein according to the present invention is depleted of cross-reactivity with endothelial cells and platelets, shortens bleeding time, and can avoid autoimmunity. In addition, DV ΔNC NS1 protein of the present invention is derived from dengue virus nonstructural protein rather than dengue virus envelope protein, and thus can be used as an inventive and practical vaccine without antibody dependent enhancement.

Although the present invention has been explained in relation to its preferred embodiment, it is to be understood that many other possible modifications and variations can be made without departing from the spirit and scope of the invention as hereinafter claimed.

What is claimed is:

1. A dengue vaccine, comprising:
N and C termini-deleted nonstructural protein ΔNC NS1 with a peptide fragment from amino acids 36 to 270, which is derived from dengue virus nonstructural protein 1 (DV NS1) with deletions of N-terminal region from amino acids 1 to 35 and C-terminal region from amino acids 271 to 352.

2. The dengue vaccine as claimed in claim 1, wherein the N and C termini-deleted nonstructural protein ΔNC NS1 has the amino acid sequence of SEQ. ID. NO. 1.

3. The dengue vaccine as claimed in claim 1, wherein the N and C termini-deleted nonstructural protein ΔNC NS1 is derived from dengue virus protein.

4. The dengue vaccine as claimed in claim 1, wherein the dengue vaccine is used to prevent dengue hemorrhagic fever or dengue shock syndrome.

5. The dengue vaccine as claimed in claim 1, wherein the amino acid sequence similarity between the N and C termini-deleted nonstructural protein ΔNC NS1 and the SEQ. ID. NO. 1 is 90% or more.

6. The dengue vaccine as claimed in claim 1, wherein the amino acid sequence similarity between the N and C termini-deleted nonstructural protein ΔNC NS1 and the SEQ. ID. NO. 1 is 95% or more.

7. A dengue vaccine-containing pharmaceutical composition, wherein the dengue vaccine comprises:
N and C termini-deleted nonstructural protein ΔNC NS1 with a peptide fragment from amino acids 36 to 270, which is derived from dengue virus nonstructural protein 1 (DV NS1) with deletions of N-terminal region from amino acids 1 to 35 and C-terminal region from amino acids 271 to 352.

8. The dengue vaccine-containing pharmaceutical composition as claimed in claim 7, wherein the N and C termini-deleted nonstructural protein ΔNC NS1 has the amino acid sequence of SEQ. ID. NO. 1.

9. The dengue vaccine-containing pharmaceutical composition as claimed in claim 7, which is used to treat or prevent dengue hemorrhagic fever or dengue shock syndrome.

10. The dengue vaccine-containing pharmaceutical composition as claimed in claim 7, wherein the amino acid sequence similarity between the N and C termini-deleted nonstructural protein ΔNC NS1 and the SEQ. ID. NO. 1 is 90% or more.

11. The dengue vaccine-containing pharmaceutical composition as claimed in claim 7, wherein the amino acid sequence similarity between the N and C termini-deleted nonstructural protein ΔNC NS1 and the SEQ. ID. NO. 1 is 95% or more.

12. A nucleotide sequence that encodes N and C termini-deleted nonstructural protein ΔNC NS1, wherein the N and C termini-deleted nonstructural protein ΔNC NS1 comprises dengue virus nonstructural protein 1 (DV NS1) from amino acids 36 to 270.

13. The nucleotide sequence as claimed in claim 12, which is SEQ. ID. NO. 2.

14. The nucleotide sequence as claimed in claim 12, which is used to produce a dengue vaccine.


SEQUENCE LISTING (4 sequences)

SEQ ID NO: 1 - protein, 235 aa, Dengue virus
PESPSKLASAIQKAHEEGICGIRSVTRLENLMWKQITPELNHILSENEVKLTIMTGDIKGIMQAGKRSLRPQPTELKYSWKTWGKAKMLPTESHNQTFLIDGPETAECPNTNRAWNSLEVEDYGFGVFTTNIWLKLREKQDVFCDSKLMSAAIKDNRAVHADMGYWIESALNDTWKIEKASFIEVKSCHWPKPHTLWSNGVLESEMIIPKNFAGPVSQHNYRPGYHTQTAGPWHL

SEQ ID NO: 2 - DNA, 705 bp, Dengue virus
ccagaatccccttcaaaactagcttcagctatccagaaagctcatgaagagggcatttgtggaatccgctcagtaacaagactggaaaatctgatgtggaaacaaataacaccagaattgaatcacattctatcagaaaatgaggtgaagttgactattatgacaggagacatcaaaggaatcatgcaggcaggaaaacgatctctgcggccccagcccactgagctgaagtattcatggaaaacatggggcaaagcgaaaatgctccctacagagtctcataaccagacctttctcattgatggccccgaaacagcagaatgccccaacacaaacagagcttggaattcgctggaagttgaagactatggctttggagtattcaccaccaatatatggctaaagttgagagaaaagcaggatgtattctgcgactcaaaactcatgtcagcggccataaaagacaacagagccgtccatgccgatatgggttattggatagaaagtgcactcaatgacacatggaagatagagaaagcctctttcatcgaagttaaaagctgccactggccaaagccacacaccctctggagtaatggagtgctagaaagtgagatgataattccaaagaatttcgctggaccagtgtcacaacacaactacagaccaggctaccatacacaaacagcaggaccatggcatcta

SEQ ID NO: 3 - protein, 352 aa, Dengue virus
DSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLPTESHNQTFLIDGPETAECPNTNRAWNSLEVEDYGFGVFTTNIWLKLREKQDVFCDSKLMSAAIKDNRAVHADMGYWIESALNDTWKIEKASFIEVKSCHWPKPHTLWSNGVLESEMIIPKNFAGPVSQHNYRPGYHTQTAGPWHLGKLEMDFDFCEGTTVVVTEDCGDRGPSLRTTTASGKLITEWCCRSCTLPPLRYRGEDGCWYGMEIRPLKEKEENLVNSLVTA

SEQ ID NO: 4 - DNA, 1056 bp, Dengue virus
gatagtggttgcgttgtgagctggaaaaacaaagaactgaagtgtggcagtgggattttcatcacagacaacgtgcacacatggacagaacaatacaagttccaaccagaatccccttcaaaactagcttcagctatccagaaagctcatgaagagggcatttgtggaatccgctcagtaacaagactggaaaatctgatgtggaaacaaataacaccagaattgaatcacattctatcagaaaatgaggtgaagttgactattatgacaggagacatcaaaggaatcatgcaggcaggaaaacgatctctgcggccccagcccactgagctgaagtattcatggaaaacatggggcaaagcgaaaatgctccctacagagtctcataaccagacctttctcattgatggccccgaaacagcagaatgccccaacacaaacagagcttggaattcgctggaagttgaagactatggctttggagtattcaccaccaatatatggctaaagttgagagaaaagcaggatgtattctgcgactcaaaactcatgtcagcggccataaaagacaacagagccgtccatgccgatatgggttattggatagaaagtgcactcaatgacacatggaagatagagaaagcctctttcatcgaagttaaaagctgccactggccaaagccacacaccctctggagtaatggagtgctagaaagtgagatgataattccaaagaatttcgctggaccagtgtcacaacacaactacagaccaggctaccatacacaaacagcaggaccatggcatctaggtaagcttgagatggactttgatttctgcgaaggaaccacagtggtggtgactgaggactgtggagatagaggaccctctttaagaacaactactgcctctggaaaactcataacagaatggtgctgccgatcttgcacattaccaccgctaagatacagaggtgaggacggatgctggtacgggatggaaatcagaccattgaaagagaaagaagagaatttggtcaactccttggtcacagcc